(19) United States
(12) Patent Application Publication
(10) Pub. No.: US 2022/0018766 A1
(43) Pub. Date: Jan. 20, 2022

(54) OPTICAL DEVICE FOR DETECTING VOLATILE COMPOUNDS AND ASSOCIATED METHOD FOR DETECTING AND QUANTIFYING VOLATILE COMPOUNDS

(71) Applicants: UNIVERSITÉ D'AIX-MARSEILLE, Marseille (FR); Centre National de la Recherche Scientifique, Paris (FR)

(72) Inventor: David Grosso, Allauch (FR)

(21) Appl. No.: 17/295,616

(22) PCT Filed: Nov. 18, 2019
(86) PCT No.: PCT/FR2019/052736

§ 371 Date: May 20, 2021

(30) Foreign Application Priority Data
Nov. 21, 2018 (FR) 1871648

(51) Int. Cl. G01N 21/55; G01N 33/00; G01N 1/44

(52) U.S. Cl. CPC G01N 21/55; G01N 1/44; G01N 33/0047

(57) ABSTRACT

An optical device, for detecting volatile compounds, comprises a sensitive reflecting element. The sensitive reflecting element comprises a substrate layer and at least one sensitive layer configured to allow volatile compounds to adsorb and desorb. An electrically conductive layer is between the substrate layer and the sensitive layer and is configured to heat the sensitive layer by Joule heating. A light source is placed to illuminate the sensitive layer. A light detector is configured to measure the light intensity reflected by the sensitive reflecting element. A computing and processing unit is also included. Also disclosed is a method for detecting and quantifying volatile compounds implementing the optical detecting device.

### CROSS-REFERENCE TO RELATED APPLICATIONS

This application is a national phase entry under 35 U.S.C. § 371 of International Patent Application PCT/FR2019/052736, filed Nov. 18, 2019, designating the United States of America and published as International Patent Publication WO 2020/104746 A1 on May 28, 2020, which claims the benefit under Article 8 of the Patent Cooperation Treaty to French Patent Application Serial No. 1871648, filed Nov. 21, 2018.

### TECHNICAL FIELD

The present disclosure relates to the field of sensors, and more particularly of sensors of volatile compounds, such as gases for example, and to the field of the use of such sensors.

### BACKGROUND

At the present time, many pollutants may be present in an atmosphere, such as, for example, the interior of a room, or even the interior of a motor-vehicle passenger compartment. These pollutants are generally in gaseous form. These gases may be colorless and odorless, or even present at levels that are too low to be directly detected by a user as he breathes. However, such gases may be harmful to health in case of regular, repeated or extended exposure.

Sensors configured to detect a particular element and to determine its concentration in an atmosphere to be tested are known in the prior art, such as, for example, that which is described in document WO 2014/189758.

Furthermore, for example from document WO 2007/019517, a detector of pollutants in a gas is known that employs a gas chromatograph oven in order to allow thermal desorption of compounds adsorbed on a selective layer. The device described in this document also employs a concentrator upstream of the measuring device, which is configured to concentrate the gas to be analyzed before it enters into the measuring device. In addition, the various desorbed elements are then analyzed. However, the pollutant detector described in this document employs elements that consume large amounts of power and requires an analyzing device in order to determine the presence of a pollutant in this gas. Thus, the pollutant detector described in this document could be improved.

Gas detectors employing a sensitive layer on which volatile compounds may adsorb are also known from documents US 2016/0084786 and FR 2871573, and from the article “Planar indium tin oxide heater for improved thermal distribution for metal oxide micromachined gas sensors,” Sensors, 2016, 16, 1612. These adsorbed compounds are then thermally desorbed and the desorption of these compounds is detected via modification of a physical property of this sensitive layer, and notably its resistance. However, detecting the modification of resistance may be complicated to do. In addition, detecting the modification of the resistance of this sensitive layer does not allow the chemical nature of the desorbed compound to be determined and it is therefore necessary to subsequently carry out complementary analyses. The various detectors described in these documents would therefore appear to require quite a long time to determine the chemical nature of the desorbed volatile compounds. In addition, the use of an ITO layer as heating element and of an electrically transduced sensitive layer requires that these two elements be separated by a non-conductive element. Such a gas detector therefore has a complex and expensive structure.

Moreover, a gas detector comprising a fixed or movable sensitive layer on which volatile compounds may adsorb is known from document FR 3046244. The adsorption or desorption of volatile compounds on or from this sensitive layer is detected via the variation in a physicochemical property of this sensitive layer. In this document, the volatile compounds are desorbed thermally. However, this document indicates that the composition of the sensitive layer may be tailored to the nature of the volatile compound that it is desired to detect. However, this document neither discloses nor suggests the way in which the chemical nature of the volatile compounds, once desorbed, may be determined or even their concentration evaluated.

### BRIEF SUMMARY

The objective of the present disclosure is to provide a detector that allows at least one volatile compound in an atmosphere to be tested to be detected and quantified that is simple to use and that delivers results in a time that is improved with respect to the detectors of the prior art.

Another objective of the present disclosure, which objective is different from the preceding one, is to provide a detector of volatile compounds that is inexpensive.

Another objective of the present disclosure, which objective is different from the preceding ones, is to provide a detector of volatile compounds that is effective even at low concentrations of volatile compounds in the atmosphere to be tested.

Another objective of the present disclosure, which objective is different from the preceding ones, is to provide a method for detecting and quantifying volatile compounds in an atmosphere to be tested that provides rapid results.

Another objective of the present disclosure, which objective is different from the preceding ones, is to provide a sensitive reflecting element that may be heated optimally without disrupting the optical transduction.

In order to at least partially achieve at least one of the aforementioned objectives, one subject of the present disclosure is an optical device for detecting volatile compounds, comprising:
a sensitive reflecting element the reflected light intensity of which varies as a function of the volatile compounds contained in an atmosphere to be tested and on their concentration, the sensitive reflecting element comprising:
   a substrate layer,
   at least one transparent and porous sensitive layer configured to allow volatile compounds contained in the atmosphere to be tested to adsorb and desorb, and
   a non-scattering and transparent electrically conductive layer sandwiched between the substrate layer and the sensitive layer and placed directly in contact with the substrate layer and with the sensitive layer, the electrically conductive layer being configured to allow the sensitive layer to be heated by Joule heating in order to allow volatile compounds adsorbed on the sensitive layer to desorb,
   at least one monochromatic or quasi-monochromatic light source placed to illuminate the sensitive layer at an incident angle,
   at least one light detector configured to measure the light intensity reflected by the sensitive reflecting element at a detection angle, and
   a computing and processing unit configured to determine the volatile compounds present in the atmosphere to be tested depending on their desorption temperatures and their concentration via variation in the light intensity reflected by the sensitive reflecting element.

Such an optical detecting device is easy to implement because it solely uses a detection of the variation in the refractive index of the sensitive reflecting element in order to identify the desorption of a volatile compound from the sensitive layer. In addition, the thermal desorption allows the chemical nature of the volatile compound to be easily and rapidly determined because each volatile compound has a specific desorption temperature that is dependent on the chemical composition of the sensitive layer. In addition, the presence of the computing and processing unit notably allows the concentration of desorbed volatile compound in the atmosphere to be tested to be determined. Thus, such an optical detecting device allows the presence of volatile compounds in an atmosphere to be tested to be simply and rapidly detected and their concentration to be determined, even at low concentrations, i.e., of the order of one ppm or even of one ppb.

The optical detecting device according to the present disclosure may furthermore comprise one or more of the following features, which may be implemented alone or in combination.

According to one aspect, the presence of one or more desorbed volatile compounds may be detected via comparison of the light intensity reflected by the reflecting sensitive element without any volatile compounds adsorbed at a given temperature with the light intensity reflected by the sensitive reflecting element just after desorption at the same temperature.

The concentration of the one or more volatile compounds detected in the atmosphere to be tested may be determined via integration of the area of peaks in variation of the reflected light intensity corresponding to the desorption of the one or more volatile compounds.

The at least one sensitive layer may have a thickness within a range from 50 nm to 2000 nm, e.g., within a range from 400 nm to 1200 nm, e.g., within a range from 500 nm to 800 nm.

The at least one sensitive layer may have an average pore size smaller than 2 nm.

According to one particular embodiment, the at least one sensitive layer may have a porosity lower than 25%.

According to one variant, the at least one sensitive layer may be made of sol-gel silica.

According to another variant, the at least one sensitive layer may be made of a xerogel.

According to one aspect of this other variant, the xerogel may comprise tetraethyl orthosilicate (TEOS).

According to another aspect of this other variant, the xerogel may comprise triethoxymethylsilane (MTEOS).

According to yet another aspect of this other variant, the xerogel may comprise phenyl-triethoxysilane (Ph-TEOS).

According to one particular embodiment, the xerogel may comprise a mixture of tetraethyl orthosilicate (TEOS), triethoxymethylsilane (MTEOS) and phenyl-triethoxysilane (Ph-TEOS).

According to one aspect, the at least one sensitive layer may have a refractive index within a range from 1.2 to 1.6, e.g., within a range from 1.3 to 1.5, at wavelengths within a range from 400 nm to 1000 nm before its exposure to the atmosphere to be tested.

Alternatively or in addition, the at least one sensitive layer may have a structure configured to increase the variations in the optical signal.

The structure of the at least one sensitive layer may comprise a diffraction grating.

The structure of the at least one sensitive layer may comprise resonant regions.

The structure of the at least one sensitive layer may comprise a photonic crystal.

The structure of the at least one sensitive layer may comprise a stack of layers.

According to a first embodiment, the sensitive reflecting element may comprise a single sensitive layer.

According to this first embodiment, the optical detecting device comprises a single light source and a single light detector.

According to a second embodiment, the sensitive reflecting element may comprise a plurality of sensitive layers.

According to this second embodiment, the optical detecting device comprises as many light sources as sensitive layers.

According to this second embodiment, the optical detecting device may comprise a single light detector when the emission wavelengths of the light sources are different.

Alternatively, the optical detecting device may comprise as many light detectors as light sources.

According to this second embodiment, the sensitive layers of the sensitive reflecting element may have perpendicular chemical affinities in order to allow a more precise chemical identification of the chemical component.

According to a first variant, the sensitive layers having perpendicular chemical affinities may be placed side-by-side.

According to a second variant, the sensitive layers having perpendicular chemical affinities may be separated from one another in the sensitive reflecting element.

According to one aspect, the substrate layer may be made of a semiconductor, e.g., of silicon, of glass, of sapphire, or of a metal.

According to one particular embodiment, the substrate layer may possess a refractive index higher than 2.5, e.g., higher than 3, at wavelengths within a range from 250 nm to 1500 nm.

The incident angle and the detection angle may be, respectively, within a range from 30° to 75° with respect to a normal to the sensitive layer.

According to one particular embodiment, the wavelength emitted by the light source is within a range from 400 nm to 1000 nm.

According to one aspect of this particular embodiment, the wavelength emitted by the light source may be monochromatic and chosen with the angle of incidence so as to coincide with the wavelength position of an inflection between two constructive and destructive interference peaks of the reflection spectrum of the sensitive reflecting element.

The electrically conductive layer may have a thickness smaller than or equal to 150 nm, e.g., within a range from 70 nm to 90 nm.

According to one aspect, the electrically conductive layer may have a resistivity within a range from 10⁻⁴ Ω·m to 10⁻⁷ Ω·m so as to allow the sensitive layer to be heated by Joule heating.

According to one aspect, the resistance of the material from which the electrically conductive layer is made is within a range from 2Ω to 200Ω, e.g., within a range from 20Ω to 50Ω.

The electrically conductive layer may be made of a transparent conductive oxide, e.g., of indium-doped tin oxide (ITO), aluminum-doped zinc oxide (AZO), gallium-doped zinc oxide (GZO), or fluorine-doped tin oxide (FTO).

According to one particular embodiment, the optical device may comprise an enclosure in which the sensitive reflecting element is placed.

The enclosure may comprise at least one aperture configured to allow the atmosphere to be tested to enter and/or exit.

Another subject of the present disclosure is a method for detecting and quantifying volatile compounds in an atmosphere to be tested implementing an optical detecting device such as described above. The detecting and quantifying method comprises the following steps:
illuminating the sensitive layer at an incident angle with the monochromatic or quasi-monochromatic light source and measuring the light intensity reflected by the sensitive reflecting element,
exposing the sensitive reflecting element to the atmosphere to be tested for a predetermined time so as to allow volatile compounds contained in this atmosphere to be tested to adsorb on the sensitive layer of the sensitive reflecting element,
heating the sensitive layer with the electrically conductive layer in order to allow the volatile compounds to be controllably desorbed from the sensitive layer,
measuring the light intensity reflected by the sensitive reflecting element during the heating step in order to determine a variation in the refractive index of the sensitive reflecting element, and
monitoring the variation in the refractive index of the sensitive reflecting element in order to determine the nature and amount of volatile compound desorbed from the sensitive layer in the heating step, the step of monitoring the variation in the refractive index of the sensitive reflecting element being carried out by the computing and processing unit.

The detecting and quantifying method, according to embodiments of the present disclosure, allows, inter alia, an adsorption of volatile compounds on the sensitive layer at room temperature. Subsequently, the adsorbed volatile compounds are detected and quantified via simple detection of a variation in the refractive index of the sensitive reflecting element, this notably allowing a rapid response to be obtained as regards the identification of the detected volatile compound or even the concentration of this volatile compound in the atmosphere to be tested.

The method for detecting and quantifying volatile compounds in an atmosphere to be tested, according to embodiments of the present disclosure, may furthermore comprise one or more of the following features, which may be implemented alone or in combination.

According to one alternative, the steps of illuminating the sensitive layer and of exposing the sensitive reflecting element to the atmosphere to be tested may be interchanged.

According to one aspect, the step of heating the sensitive layer may be carried out via controlled heating at a heating rate within a range from 1° C./s to 20° C./s.

According to one particular embodiment, the nature of the volatile compound desorbed during the heating step is determined via comparison of the refractive index of the reflecting sensitive element at a given temperature with respect to the refractive index of this sensitive reflecting element without any adsorbed compound at the same temperature.

According to one alternative, the nature of the desorbed volatile compound is determined by plotting the curve of variation in refractive index of the sensitive reflecting element as a function of the temperature of the electrically conductive layer and by determining the desorption temperature, which corresponds to the temperature at which the value of the derivative of this curve is zero, by solving the equation:

$$dN 1 / dT = 0$$

with:
   T: the temperature of the electrically conductive layer in ° C., and
   N1: the value of the refractive index of the sensitive reflecting element.

According to another alternative, the amount of volatile compound desorbed is determined by comparing the variation in refractive index before and after heating.

According to this other alternative, the amount of volatile compound desorbed is proportional to the equation:

N2−N1
with:
   N1: the value of the refractive index of the sensitive reflecting element with the adsorbed volatile compound, and
   N2: the value of the refractive index of the sensitive reflecting element without the volatile compound.

According to one aspect, the step of measuring the light intensity reflected by the sensitive layer may be carried out at a time interval within a range from 0.2 seconds to 5 seconds.

The desorbed volatile compounds may be quantified via integration of an area of a peak of variation in the refractive index of the sensitive reflecting element corresponding to the desorption of the corresponding volatile compound.

According to one variant, the volatile compound, in the case where there is a mixture of volatile compounds, may be identified via deconvolution of the refractive index of the sensitive reflecting element during the desorption of this volatile compound.

According to one particular embodiment, the wavelength of the light source may be chosen so as to coincide with the wavelength position of an inflection between two constructive and destructive interference peaks of the reflection spectrum of the sensitive reflecting element.

### BRIEF DESCRIPTION OF THE DRAWINGS

Other features and advantages of embodiments of the present disclosure will become more clearly apparent on reading the following nonlimiting description, which is given by way of illustration, and the accompanying drawings in which:

FIG. 1 is a schematic representation in perspective of an optical detecting device;

FIG. 2 is a schematic representation in cross section of a sensitive reflecting element of the optical detecting device of FIG. 1;

FIG. 3A is a schematic representation in cross section of a sensitive reflecting element of the optical detecting device of FIG. 1 according to a first embodiment;

FIG. 3B is a schematic representation in cross section of a sensitive reflecting element of the optical detecting device of FIG. 1 according to a second embodiment;

FIG. 4A is a schematic representation in cross section of the optical detecting device of FIG. 1 according to a first variant;

FIG. 4B is a schematic representation in cross section of the optical detecting device of FIG. 1 according to a second variant;

FIG. 4C is a schematic representation in cross section of the optical detecting device of FIG. 1 according to a third variant;

FIG. 5 is a schematic representation of a flow chart illustrating various steps of a method for detecting and quantifying volatile compounds in an atmosphere to be tested employing the optical detecting device of FIG. 1;

FIG. 6A is a schematic representation of a sensitive layer of the optical detecting device of FIG. 1 during the method for detecting and quantifying volatile compounds;

FIG. 6B is a schematic representation of curves obtained after processing during a desorption of volatile compounds from the sensitive layer of FIG. 6A;

FIGS. 7A to 7F are schematic representations of curves of variation in refractive index that are obtained during desorptions of various isolated volatile compounds for a sensitive layer having one particular chemical composition; and

FIGS. 8A to 8C are schematic representations of curves of variation in refractive index that are obtained during desorption of isolated volatile compounds contained in a given atmosphere to be tested for sensitive layers having different chemical compositions.

Identical elements in the various figures have been designated with the same references.

### DETAILED DESCRIPTION

The following embodiments are examples. Although the description refers to one or more embodiments, this does not necessarily mean that each reference relates to the same embodiment, or that the features apply only to a single embodiment. Single features of various embodiments may also be combined and/or interchanged to form other embodiments.

In the present description, certain elements or parameters may be indexed (e.g., first element or second element and first parameter and second parameter or even first criterion and second criterion, etc.). In this case, it is simply a question of an indexation that is used to differentiate and denote elements, parameters or criteria that are similar but not identical. This indexation does not imply a priority of one element, parameter or criterion with respect to another and such denominations could easily be interchanged without departing from the scope of the present description. This indexation also does not imply, for example, an order in time that such or such criteria must be estimated.

In the present description, by “volatile compound” what is meant is a compound that vaporizes or evaporates easily under standard temperature and pressure conditions (i.e., 25° C. and 1 bar) such as defined by the International Union of Pure and Applied Chemistry. In the present description, a volatile compound may be of organic nature; it may be, for example, a question of a volatile organic compound (also known by the acronym VOC), or even of a volatile compound of inorganic nature, such as water, for example.

In addition, in the following description, by “transparent” what is meant is a material that is preferably colorless, through which light may pass with a maximum intensity absorption of 10% at wavelengths, in particular, within a range from 280 nm to 1300 nm.

Moreover, in the following description, by “xerogel” what is meant is a material comprising a macromolecular network of vitreous oxides that is manufactured using a sol-gel process.

Below, in the following description, by “quasi-monochromatic” what is meant is a wave the spectrum of which occupies only a very narrow wavelength band; the spectrum of this wave may occupy, for example, a wavelength band narrower than 2 nm.

### Optical Detecting Device

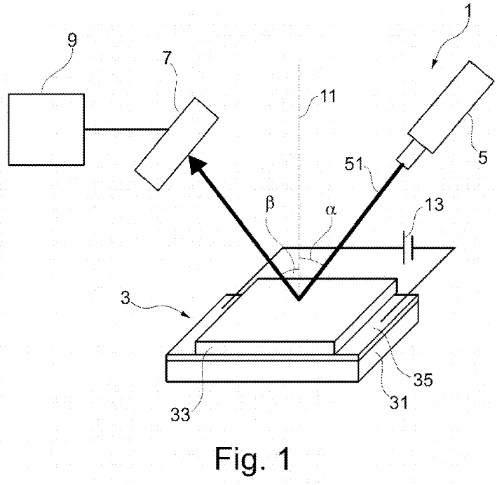

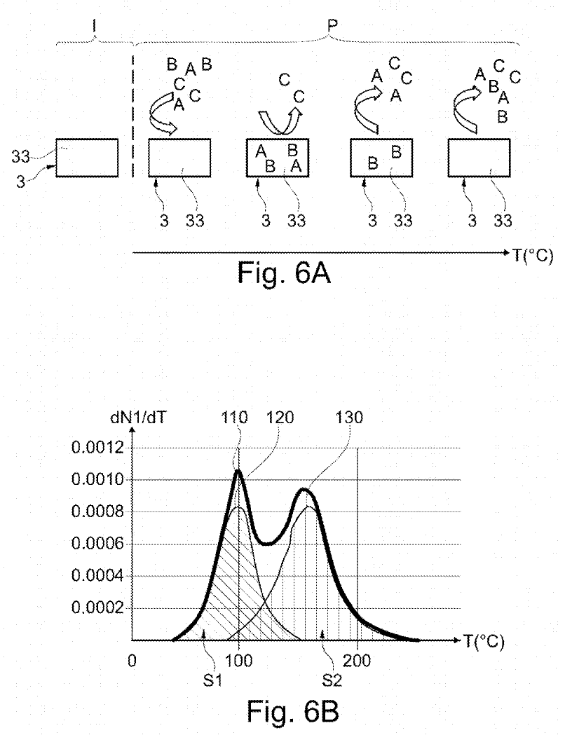

In FIGS. 1 to 4C, an optical device 1 for detecting volatile compounds A, B, C (shown in FIG. 6A) is illustrated. The optical detecting device 1 comprises a sensitive reflecting element 3, at least one monochromatic or quasi-monochromatic light source 5, at least one light detector 7, and a computing and processing unit 9.

The sensitive reflecting element 3 is intended to be placed in an atmosphere to be tested so that at least some of the volatile compounds present in this atmosphere to be tested are adsorbed by this sensitive reflecting element 3, in order to make it possible to detect their presence and to determine their concentration on their desorption. Thus, the intensity of the reflection of the sensitive reflecting element 3 is intended to vary as a function of the volatile compounds A, B, C contained in the atmosphere to be tested that are adsorbed by this sensitive reflecting element 3 and of their concentration. The sensitive reflecting element 3 comprises a substrate layer 31, at least one sensitive layer 33, and an electrically conductive layer 35 sandwiched between the substrate layer 31 and the sensitive layer 33. Moreover, the electrically conductive layer 35 is configured to heat the sensitive layer 33 by Joule heating.

The substrate layer 31 may be made of a semiconductor, e.g., silicon, of glass, of sapphire, or of a metal. Furthermore, the substrate layer 31 possesses a refractive index higher than 2.5, e.g., higher than 3, at wavelengths within a range from 250 nm to 1500 nm. In addition, the substrate layer 31 may be reflective for example because of a mirror-type metal coating on one of its faces, which face is placed facing the electrically conductive layer 35.

Moreover, the at least one sensitive layer 33 is porous and transparent. This sensitive layer 33 is configured to allow at least some of the volatile compounds A, B, C (shown in FIG. 6A) contained in the atmosphere to be tested to adsorb and desorb. This sensitive layer 33 may have a particular chemical affinity with certain volatile compounds, such as, for example, halogen-containing derivatives such as chloroform for example, the aldehydes or ketones such as acetylacetone for example, saturated or unsaturated linear or even cyclic hydrocarbons such as hexane or toluene for example, and/or alcohols such as ethanol or ethylene glycol for example. More particularly, the adsorption of the volatile compounds on this sensitive layer 33 corresponds to a physisorption, i.e., this adsorption is achieved by virtue of low-energy forces and in particular by virtue of Van der Waals forces. Thus, it is possible to promote or not the adsorption of certain families of volatile compounds by modifying the nature and composition of this sensitive layer 33. In addition, it is also possible to modify the selectivity of this sensitive layer 33 to a particular family of volatile compounds A, B, C by modifying the steric bulk of the pores or even their chemical affinity. The at least one sensitive layer 33 may have an average pore size smaller than 2 nm and a porosity lower than 25%. Such properties for the sensitive layer 33 endow this sensitive layer 33 with adsorption properties that are suitable for many volatile compounds A, B, C. Specifically, in the case where the pores are too large in size, it is possible for certain compounds to desorb easily and this may compromise the precision of the measurement of these volatile compounds. The average diameter of the pores may be determined using a known volumetry method, for example, a method described in detail in the article “Porosity and mechanical properties of mesoporous thin films assessed by environmental ellipsometric porosimetry” Cedric Boissière et al., Published in American Chemical Society, 2005. Langmuir: the ACS journal of surfaces and colloids 2005, 21, 12362-71. In addition, the sensitive layer 33 has an accessible pore surface area larger than 140 cm²/cm². Furthermore, the at least one sensitive layer 33 may have a refractive index within a range from 1.2. to 1.6, e.g., within a range from 1.3 to 1.5, at wavelengths within a range from 400 nm to 1000 nm before its exposure to the atmosphere to be tested. Tests carried out by the inventors have shown that a sensitive layer 33 having such properties allows a sensitivity within a range from 10⁻⁴ to 10⁻⁵ optical units per ppm of volatile compound A, B, C to be obtained.

The at least one sensitive layer 33 may be made of sol-gel silica, or even be a xerogel. By virtue of the use of sol-gel processes, it is possible to easily control the formation of this sensitive layer 33 and, e.g., its thickness e (shown in FIGS. 3A and 3B). More particularly, the at least one sensitive layer 33 has a thickness e within a range from 50 nm to 2000 nm, e.g., within a range from 400 nm to 1200 nm, e.g., within a range from 500 nm to 800 nm. The thickness e of the sensitive layer 33 allows quite short response times to be obtained. Specifically, if the thickness e of the sensitive layer 33 is too large, this leads to a quite long equilibrium time, this possibly being considered detrimental by a user of the optical detecting device 1. In addition, the thickness e of the sensitive layer 33 allows an inflection point of maximum slope to be placed at a measurement wavelength corresponding to the emission wavelength of the light source 5. In addition, such a thickness e of the sensitive layer 33 allows the latter to have a uniform temperature during the heating thereof. Specifically, if the thickness e of the sensitive layer 33 is too large, then the temperature of the latter will not be uniform right through its thickness e during the heating thereof, this possibly being detrimental to the measurements carried out with the optical detecting device 1.

Moreover, the at least one sensitive layer 33 does not comprise any structuring agents, i.e., chemical species of mineral or organic nature about which the material from which the sensitive layer 33 is made could organize.

Furthermore, in the case where the sensitive layer 33 is formed by a xerogel, this xerogel may comprise tetraethyl orthosilicate (TEOS), triethoxymethyisilane (MTEOS), or even phenyl-triethoxysilane (Ph-TEOS). According to one particular embodiment described in more detail below, the xerogel may comprise a mixture of tetraethyl orthosilicate (TEOS), triethoxymethylsilane (MTEOS) and phenyl-triethoxysilane (Ph-TEOS).

Moreover, the electrically conductive layer 35 of the sensitive reflecting element 3 is non-scattering, i.e., it does not scatter light, and transparent at wavelengths within a range from 400 nm to 1000 nm. Specifically, if this electrically conductive layer 35 scattered light, this could potentially be detrimental to the precision of the measurement taken. Specifically, if the electrically conductive layer 35 is scattering, some of the light ray 51 may be scattered, this possibly being detrimental to the intensity of the signal once reflected and making the signal difficult to analyze with the light detector because of the initial signal being subject to masking related to scattering of the signal in the electrically conductive layer 35. In addition, if this electrically conductive layer 35 is opaque, in this case, it is not possible to obtain a reflected ray and therefore to detect the latter with the light detector 7. This electrically conductive layer 35 is placed directly in contact with the substrate layer 31 and with the sensitive layer 33. This electrically conductive layer 35 is configured to allow the sensitive layer 33 to be heated by Joule heating in order to allow desorption of the volatile compounds A, B, C adsorbed on the sensitive layer 33. To this end, the electrically conductive layer 35 may have a resistivity within a range from 10⁻⁴ Ω·m to 10⁻⁷ Ω·m. Furthermore, the component of this electrically conductive layer 35 may have a resistance within a range from 2Ω to 200Ω, e.g., within a range from 20Ω to 50Ω, so as to allow the sensitive layer 33 to be heated by Joule heating. Placing this electrically conductive layer 35 directly in contact with the sensitive layer 33 allows the latter to be effectively heated and limits the time required to desorb the volatile compounds A, B, C adsorbed on the sensitive layer 33. In addition, the electrically conductive layer 35 may have a thickness s (shown in FIGS. 3A and 3B) smaller than or equal to 150 nm, e.g., within a range from 70 nm to 90 nm. The thickness s of this electrically conductive layer 35 may be adjusted so as not to disrupt the optical readout and also so as to adjust the resistivity of this electrically conductive layer 35 in order to tailor the required electrical power to the heating conditions.

Moreover, the electrically conductive layer 35 may, for example, be made of a transparent conductive oxide (TCO), e.g., of tin-doped indium oxide (ITO), aluminum-doped zinc oxide (AZO), gallium-doped zinc oxide (GZO), or of fluorine-doped tin oxide (FTO). Such compounds have resistivity properties that are compatible with those required to allow the sensitive layer 33 to be heated by Joule heating. Such a composition of the electrically conductive layer 35 (layer made of a transparent conductive oxide) allows optical transduction via direct reflection of a light beam (e.g., light ray 51) emitted by the light source 5. Furthermore, transparent conductive oxides (TCOs) are good candidates for sol-gel production, and therefore for the formation of the sensitive layer 33. According to this particular embodiment, the substrate layer 31 comprises a mirror-type metal coating on its face placed in contact with the electrically conductive layer 35 in order to improve the intensity of the reflected light ray.

Thus, the sensitive reflecting element 3 has a small thickness, this allowing its placement at measurement sites or even its transport to be facilitated. In addition, the sensitive reflecting element 3 may be placed at measurement sites without requiring other elements of the optical detecting device 1 to be present, the elements being necessary only during the study of the desorption of any volatile compounds A, B, C adsorbed on the sensitive layer 33.

Moreover, the light source 5 is placed to illuminate the sensitive layer 33 at an incident angle α. According to the particular embodiment of FIG. 1, the wavelength emitted by the light source 5 is within a range from 400 nm to 1000 nm. Moreover, the wavelength emitted by the light source 5 may be monochromatic and chosen with the angle of incidence α so as to coincide with the wavelength position of an inflection between two constructive and destructive interference peaks of the reflection spectrum of the sensitive reflecting element 3. The thickness e of the sensitive layer 33 is chosen to be large enough that the slopes of the regions of inflection of the reflection spectrum are accentuated without causing the number of interferences to exceed a value that would make alignment too tricky.

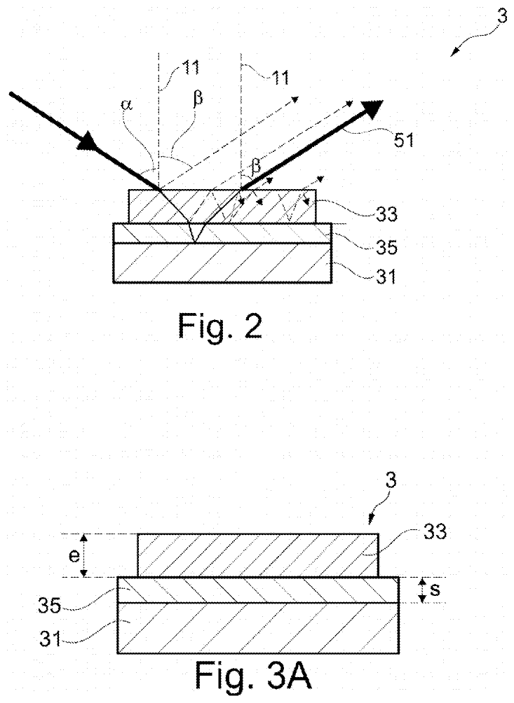

In addition, the light detector 7 is configured to measure the light intensity reflected by the sensitive reflecting element 3 at a detection angle β. This light detector 7 may be, for example, a camera or any other element able to detect the light ray 51 reflected by the sensitive reflecting element 3. According to the particular embodiment of FIG. 1, the incident angle α and the detection angle β are, respectively, within a range from 30° to 75° with respect to a normal 11 to the sensitive layer 33. This normal 11 is an imaginary line placed perpendicular to the sensitive layer 33. Furthermore, the incident angle α and the detection angle β may be equal in the case of specular reflection, but this is not essential. In FIG. 2, the optical path of the light ray 51 in the sensitive layer 33 and in the electrically conductive layer 35 has been shown. As shown in FIG. 2, the incident angle α and the reflection angle β are determined with respect to the normal 11 to the sensitive layer 33. In FIG. 2, the representation of the optical path has been intentionally exaggerated in order to allow the incident angle α and the detection angle β and the various deviations of this light ray 51 that occur at the interfaces with the sensitive layer 33 and electrically conductive layer 35 to be correctly identified.

According to a simplified approach based on fresnel's equations and not taking into account the presence of the substrate layer 31, the following supposition may be made:

$$Rs = {\vert \frac{{n}_{1} cos ( α ) - {n}_{2} cos ( β )}{{n}_{1} cos ( α ) + {n}_{2} cos ( β )} \vert}^{2}$$

$$Rp = {\vert \frac{{n}_{1} cos ( β ) - {n}_{2} cos ( α )}{{n}_{1} cos ( β ) + {n}_{2} cos ( α )} \vert}^{2}$$

$$R = 1 / 2 ( Rs + Rp )$$

where
   Rs is the reflectivity for incident light of s-polarization,
   Rp is the reflectivity for incident light of p-polarization,
   R is the reflectivity in the case of unpolarized light,
   α is the angle of the incident light ray 51,
   β is the angle of the reflected light ray 51,
   n₁ is the refractive index of air (n₁=1=constant), and
   n₂ is the refractive index of the sensitive reflecting element 3, which varies as a function of the adsorption of volatile compounds A, B, C contained in the atmosphere to be tested by the sensitive layer 33.

Given that in this formula only n₂ varies as a function of the adsorption of volatile compounds A, B, C contained in the atmosphere to be tested by the sensitive layer 33, n₁, α and β remaining constant, it is possible therefore to deduce therefrom that reflectance decreases linearly with an increase in the refractive index n₂. It turns out that the refractive index n₂ of the sensitive reflecting element 3 varies linearly with the amount of volatile compounds A, B, C adsorbed by the sensitive layer 33.

Returning to FIG. 1, the computing and processing unit 9 is configured to determine the volatile compounds A, B, C present in the atmosphere to be tested depending on their desorption temperatures and their concentration via variation in the light intensity reflected by the sensitive reflecting element 3. The computing and processing unit 9 allows the presence of one or more volatile compounds A, B, C described from the sensitive layer 33 to be detected via comparison of the light intensity reflected by the sensitive reflecting element 3 at a given temperature with the light intensity reflected by this sensitive reflecting element 3 before its exposure to the atmosphere to be tested at the same temperature, as is described in more detail below. In addition, the concentration of the one or more volatile compounds A, B, C detected in the atmosphere to be tested is determined via integration of the area of each peak of variation in the refractive index of the sensitive reflecting element 3, corresponding to the desorption of volatile compounds A, B, C. This integration may notably be carried out after calibration of the computing and processing unit 9.

Thus, the optical detecting device 1 allows the presence of one or more volatile compounds A, B, C in an atmosphere to be tested to be detected easily via a simple variation in the intensity of reflection of a light ray 51 allowing a variation in the refractive index of the sensitive reflecting element 3 to be determined. This detection may therefore be rapid. Specifically, since the transduction is of optical-reflection type, volatile compounds A, B, C are detected by measuring the variation in the intensity reflected at a single wavelength, this variation being induced by the increase in the refractive index associated with the adsorption of the volatile compound A, B, C in the sensitive layer 33. In addition, it is simple and rapid to use thermal desorption to detect and quantify the desorbing volatile compound A, B, C that was initially present in the atmosphere to be tested. In addition, the various components of the sensitive reflecting element 3 are quite inexpensive, this allowing the manufacturing cost of this optical detecting device 1 to be limited.

Moreover, the sensitivity of the optical detecting device 1 may be enhanced by judiciously choosing in particular the thickness e of the sensitive layer 33 on the one hand and the wavelength combined with the angle of incidence α on the other hand. Specifically, above simplified Fresnel's equations were presented with a view to explaining the basis of operation of the optical detecting device 1.

Alternatively or in addition, the at least one sensitive layer 33 may have a structure configured to increase the variations in the optical signal in order to improve the sensitivity of this optical detecting device 1. The structure of the at least one sensitive layer 33 may comprise a diffraction grating, resonant regions, a photonic crystal, or even a stack of layers.

According to the particular embodiment of FIG. 3A, the sensitive reflecting element 3 may comprise a single sensitive layer 33. This sensitive layer 33 may be selective to a single family of chemical compounds or even to a plurality of families of chemical compounds. When the sensitive reflecting element 3 comprises a single sensitive layer 33, the optical detecting device 1 comprises a single light source 5 and a single light detector 7. In this particular embodiment, the electrically conductive layer 35 has a thickness s of 80 nm, an area of 5×5 mm² and a resistance of 30Ω. Such an electrically conductive layer 35, when it is powered by the generator 13 (shown in FIG. 1) at a voltage of 4.5 V, allows the sensitive layer 33 to reach a temperature of 80° C. in 25 seconds; or indeed when the generator 13 delivers 12 V to this electrically conductive layer 35, the temperature of the sensitive layer 33 reaches a temperature of 300° C. The presence of a single sensitive layer 33 in the sensitive reflecting element 3 allows its manufacture to be facilitated. However, when volatile compounds A, B, C have similar temperatures of desorption from this sensitive layer 33, it may be possible to be unable to distinguish these volatile compounds A, B, C, as is explained below. Such situations are quite rare but may occur.

In order to mitigate this eventuality, and as shown in FIG. 3B, the sensitive reflecting element 3 may comprise a plurality of sensitive layers 33 placed adjacent one another on the electrically conductive layer 35. In order to allow a discrimination between volatile compounds A, B, C having a similar, or even identical, desorption temperature, this possibly occurring with the sensitive layer 33 of FIG. 3A, the sensitive layers 33a, 33b, 33c of the sensitive reflecting element 3 may have perpendicular chemical affinities, thus allowing a more precise chemical identification of the volatile compounds A, B, C. By “perpendicular chemical affinities” what is meant is the fact that one of the sensitive layers 33a, 33b, 33c has a chemical affinity that is high, or even exclusive, with respect to a first family of chemical compounds and that is negligible, or even zero, with respect to a second family of chemical compounds, and that another of the sensitive layers 33a, 33b, 33c has a chemical affinity that is high, or even exclusive, with respect to this second family of chemical compounds and that is negligible, or even zero, with respect to this first family of chemical compounds.

In the particular representation of FIG. 3B, the sensitive reflecting element 3 comprises three sensitive layers 33a, 33b, 33c. The sensitive layers 33a, 33b, 33c having perpendicular chemical affinities correspond to the sensitive layers 33a and 33b, and they are therefore placed side-by-side. Alternatively, the sensitive layers 33a, 33b, 33c having perpendicular chemical affinities correspond to the sensitive layers 33a and 33c, and they are therefore separated from each other in the sensitive reflecting element 3. Obviously, the compositions of the sensitive layers 33a, 33b, 33c are different from one another and the temperatures of desorption of the volatile compounds A, B, C from these sensitive layers 33a, 33b, 33c may be different from one another depending on the sensitive layer 33a, 33b, 33c in question, as is described in more detail below. Specifically, by changing the chemical composition of the sensitive layers 33a, 33b, 33c, the chemical affinity of these sensitive layers 33a, 33b, 33c to a given volatile compound A, B, C is changed and the temperature of desorption of a given volatile compound A, B, C, from sensitive layers 33a, 33b, 33c having different chemical compositions, may be changed. In this particular embodiment, the optical detecting device 1 comprises as many light sources 5 as different sensitive layers 33a, 33b, 33c, each light source 5 directing one light ray 51 (e.g., one light beam) in the direction of one specific sensitive layer 33a, 33b, 33c. In order to monitor the variation in the optical property of the sensitive layer 33 as a function of temperature during the desorption of the volatile compounds A, B, C, the optical detecting device 1 may comprise a single light detector 7 in the case where the various light sources 5 emit light rays 51 (e.g., light beams) of different wavelengths. Alternatively, the optical detecting device 1 may comprise as many detecting devices (e.g., light detectors 7) as light sources 5, each detecting device (e.g., light detector 7) being associated with one particular light source 5 and with one particular sensitive layer 33a, 33b, 33c. A sensitive reflecting element 3 according to the particular embodiment of FIG. 3B may, for example, form an artificial nose.

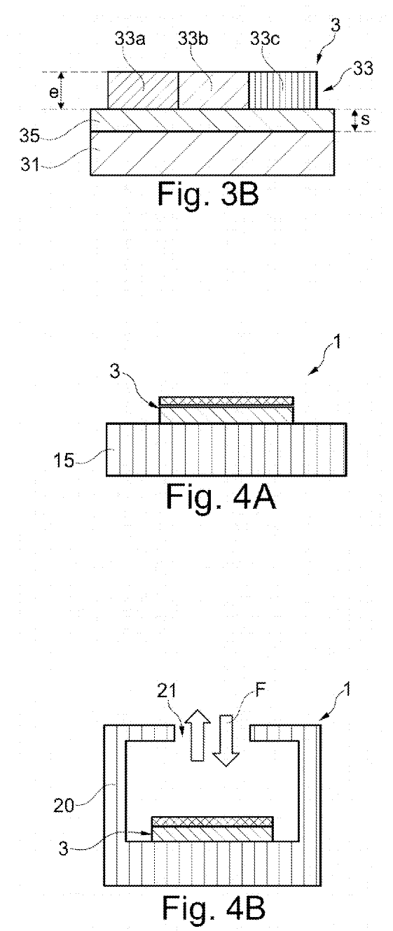

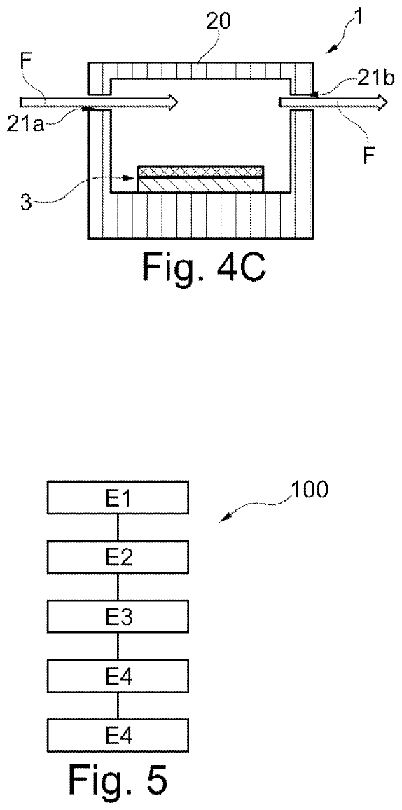

In FIGS. 4A to 4C, various variants of the optical detecting device 1 have been shown.

In FIG. 4A, the sensitive reflecting element 3 may be placed on a base 15 and left in the open air in order to allow adsorption of volatile compounds A, B, C present in the atmosphere surrounding this optical detecting device 1. The base 15 shown in FIG. 4A may correspond to an element on which the sensitive reflecting element 3 is placed, such as a table, for example. This base 15 may also correspond to an adhesive configured to allow this sensitive reflecting element 3 to be fastened to any type of holder. Thus, this base 15 may be an additional element added to the sensitive reflecting element 3 or be an element separate from the optical detecting device 1.

In FIGS. 4B and 4C, the optical detecting device 1 may comprise an enclosure 20 in which the sensitive reflecting element 3 is placed. The presence of this enclosure 20 may allow handling and movement of the sensitive reflecting element 3 to be facilitated. Specifically, the presence of this enclosure 20 may prevent potential contamination of the sensitive layer 33 with compounds potentially present on the hands of a handler. The enclosure 20 comprises at least one aperture 21 configured to allow the atmosphere to be tested to enter and/or exit in the form of a gas flow F. More particularly, the optical detecting device 1 of FIG. 4B comprises a single aperture 21 in order to allow the gas flow F to enter and exit. Such an optical detecting device 1 may also be used to detect the presence of certain volatile compounds in a free atmosphere, for example as may be the case with the air present in a room or in a motor-vehicle passenger compartment. Furthermore, in the particular embodiment of FIG. 4C, the enclosure 20 comprises a first aperture 21a configured to allow the gas flow F to enter into the enclosure 20 and a second aperture 21b configured to allow this gas flow F to exit from the enclosure 20. In the particular embodiment of FIG. 4C, the airflow F is generated inside the enclosure 20 using a fan (not shown) placed inside this enclosure 20. The optical detecting device 1 of FIG. 4C may, for example, be used to detect the presence of certain volatile compounds present in a gas contained in a canister, for example, or even in the atmosphere of a room or of a vehicle passenger compartment. In the embodiments of FIGS. 4B and 4C, the light source 5 and the light detector 7 may be placed in this enclosure 20 so as to obtain a portable optical detecting device 1. In such a configuration, the refractive index of the sensitive layer 33 may be measured and monitored in situ during the heating of this sensitive layer 33 by the electrically conductive layer 35. Alternatively, the enclosure 20 may contain only the sensitive reflecting element 3, the other elements being placed outside the enclosure 20. According to yet another alternative, the sensitive reflecting element 3 may be removable from this enclosure 20 in order to allow the volatile compounds A, B, C adsorbed on the sensitive reflecting element 3 to be desorbed and therefore detected and quantified.

The inventors have observed that, depending on the presence and on the configuration of the enclosure 20, the rates of adsorption of the volatile compounds A, B, C are not identical. More particularly, the configuration of the enclosure 20 comprising the first and second apertures 21a, 21b, i.e., the configuration such as shown in FIG. 4C, allows a more rapid adsorption of the volatile compounds on the sensitive layer 33, and the configurations shown in FIGS. 4A and 4B provide a substantially equal adsorption rate that is however lower than that of the configuration of the enclosure 20 of FIG. 4C.

### Detecting and Quantifying Method

In FIG. 5, a flowchart illustrating a method 100 for detecting and quantifying volatile compounds A, B, C (shown in FIG. 6A) in an atmosphere to be tested employing the optical detecting device 1 described above has been shown.

The detecting and quantifying method 100 comprises a step E1 of illuminating the sensitive layer 33 at an incident angle α with the monochromatic or quasi-monochromatic light source 5 (these being shown in FIG. 1) and measuring the light intensity reflected by the sensitive reflecting element 3. This illuminating step E1 allows an initial reflectance of the light ray 51 before any adsorption of volatile compounds A, B, C on the sensitive layer 33 to be determined.

The detecting and quantifying method 100 then implements a step E2 of exposing the sensitive reflecting element 3 to the atmosphere to be tested for a predetermined time so as to allow volatile compounds A, B, C contained in this atmosphere to be tested to adsorb on the sensitive layer 33 of the sensitive reflecting element 3. In this exposing step E2, the sensitive layer 33 of the sensitive reflecting element 3 is also illuminated in order to be able to observe any adsorption of volatile compounds A, B, C on this sensitive layer 33. Specifically, the refractive index of the sensitive reflecting element 3 increases or decreases on the adsorption of volatile compounds A, B, C. The illumination of the sensitive layer 33 in this exposing step E2 therefore makes it possible to determine whether volatile compounds A, B, C have been adsorbed by the sensitive layer 33.

According to one alternative, the steps E1 and E2 of illuminating the sensitive layer 33 and of exposing the sensitive reflecting element 3 to the atmosphere to be tested may be inverted. More particularly, the step E2 of exposing the sensitive reflecting element 3 to the atmosphere to be tested may be implemented prior to the step E1 of illuminating the sensitive layer 33.

The detecting and quantifying method 100 then implements a step E3 of heating the sensitive layer 33 via the electrically conductive layer 35 in order to allow the volatile compounds A, B, C to be desorbed from the sensitive layer 33. According to one particular embodiment, the step E3 of heating the sensitive layer 33 is carried out via controlled heating at a heating rate within a range from 1° C./s to 20° C./s.

The detecting and quantifying method 100 comprises a step E4 of measuring the light intensity reflected by the sensitive reflecting element 3 during the heating step E3 in order to determine a variation in the refractive index of this sensitive reflecting element 3. The step E4 of measuring the light intensity reflected by the sensitive reflecting element 3 may, for example, be carried out at time intervals within a range from 0.2 seconds to 5 seconds. Specifically, the modification of the refractive index of the sensitive reflecting element 3 allows the adsorption of volatile compounds A, B, C by the sensitive layer 33 of the latter or even the desorption of volatile compounds A, B, C from the sensitive layer 33 of this sensitive reflecting element 3 to be detected.

Lastly, the detecting and quantifying method 100 comprises a step E5 of monitoring the variation in the refractive index of the sensitive reflecting element 3 in order to determine the chemical nature and the amount of volatile compound A, B, C desorbed from the sensitive layer 33 during the heating step E3. This step E5 of monitoring the variation in the refractive index of the sensitive reflecting element 3 is carried out by the computing and processing unit 9. More particularly, in this step E5 of monitoring the variation in the refractive index of the sensitive reflecting element 3, the computing and processing unit determines the nature of the volatile compound A, B, C desorbed during the heating step E3 by taking the derivative of the signal with respect to temperature and by comparing the value of the temperature at which the derivative in question is zero to temperatures present in a database. Furthermore, the desorbed volatile compound A, B, C is quantified via integration of the area of the various peaks of variation in the reflected light intensity corresponding to the desorption of the desorbed volatile compound. This integration may be carried out after calibration of the computing and processing unit 9.

The inventors have determined, in the course of various tests, that all the volatile compounds A, B, C potentially adsorbed on the sensitive layers 33 are completely desorbed when this sensitive layer 33 reaches a temperature higher than or equal to 250° C. If a heating rate within a range from 1° C./s to 20° C./s is used, this detecting and quantifying method 100 is observed to provide short response times.

In FIG. 6A, a schematic of the detecting and quantifying method 100 described with reference to FIG. 5 has been shown. In this schematic, the sensitive layer 33 of the sensitive reflecting element 3 has been shown in an initial state I and in a polluted state P during the detecting and quantifying method 100. When the sensitive layer 33 is in its initial state I, no volatile compound A, B, C is adsorbed on the latter. In order to allow the measurements to be calibrated, it is possible to carry out measurements of the refractive index of this sensitive layer 33 without any elements adsorbed thereon as a function of its temperature, by heating this sensitive layer 33 by activating the generator 13 (shown in FIG. 1) so that it delivers energy to the electrically conductive layer 35 and heats the sensitive layer 33 by Joule heating.

The sensitive layer 33 is then exposed to the atmosphere to be tested, which potentially contains volatile compounds A, B, C at least some of which are intended to adsorb on the sensitive layer 33. The sensitive layer 33 is typically exposed to the atmosphere to be tested at room temperature, i.e., about 25° C. This exposure occurs for a predetermined time in order to allow at least some of the volatile compounds A, B, C to adsorb on this sensitive layer 33. This exposure time may be within a range from a few minutes, typically 10 minutes to 30 minutes, to several days, typically five days or six days. In the example shown in FIG. 6A, the sensitive layer 33 is specific to the volatile compounds A and B, which may adsorb on the latter, whereas the volatile compound C has a negligible, or even zero, chemical affinity with this sensitive layer 33. The volatile compound C is therefore not adsorbed on the latter. The sensitive layer 33 is then heated by Joule heating on the supply of the electrically conductive layer 35 with current by the generator 13. During this heating, the chemical species A and B that are adsorbed on this sensitive layer 33 will desorb at different temperatures. This differential desorption allows the presence of the volatile compounds A and B in this atmosphere to be tested to be detected and these volatile compounds A and B to be identified by virtue of their desorption temperature. It may be observed that the sensitive layer 33 allows a dual selectivity to be obtained with respect to the volatile compounds A, B, C: a first selectivity related to the adsorption of the volatile compound on this sensitive layer 33, and a second selectivity related to its temperature of desorption from this sensitive layer 33. Such a measuring (e.g., detecting) and quantifying method 100 therefore allows a high measurement sensitivity to be obtained.

Specifically, as is illustrated in FIG. 6B, the volatile compounds A and B will desorb at different temperatures from this sensitive layer 33. This differential desorption as a function of the temperature of the sensitive layer 33 is related to the chemical affinity that these chemical compounds A and B have with the sensitive layer 33 and to their heat of desorption. Specifically, the higher the heat of desorption and chemical affinity between the volatile compound A, B, C and the sensitive layer 33, the higher the desorption temperature of this volatile compound A, B, C. Thus, in the schematic illustrated in FIG. 6A, the volatile compound B has a higher chemical affinity with the sensitive layer 33 than the volatile compound A, which will desorb at a lower temperature as illustrated in FIG. 6B.

In FIG. 6B, the nature of the desorbed volatile compound is determined by plotting the curve of variation in refractive index of the sensitive reflecting element 3 as a function of the temperature of the electrically conductive layer 35 and by determining the desorption temperature, which corresponds to the temperature at which the value of the derivative of this curve is zero, by solving the equation:

dN1/dT=0
with:
   T: the temperature of the electrically conductive layer 35 in ° C., and
   N1: the value of the refractive index of the sensitive reflecting element 3.

More particularly, in FIG. 6B, the curve 110 corresponds to the plot of the curve satisfying this equation. The computing and processing unit 9 (shown in FIG. 1) then allows the volatile compound A, B, C, in the case where there is a mixture of volatile compounds A, B, C as is the case here, to be identified. The volatile compound A, B, C is identified via mathematical deconvolution of the derivative of the light intensity reflected by the sensitive reflecting element 3 as a function of temperature during the desorption of this gas. More particularly, these deconvolutions are illustrated by the curve 120, which is representative of the volatile compound A during its desorption, and the curve 130, which is representative of the volatile compound B during its desorption. By interpreting the various curves of FIG. 6B, it is possible to deduce that compound A desorbs from the sensitive layer 33 at a temperature of about 94° C. and that compound 13 desorbs from this sensitive layer 33 at a temperature of about 161° C. The determination of these desorption temperatures, which are dependent on the composition of the sensitive layer 33, allows the nature of the desorbed volatile compound A, B, C to be determined. These various desorption temperatures as a function of the composition of the sensitive layer 33 may, for example, be listed in tables.

Furthermore, the integration of an area S1 placed under the curve 120, corresponding to the area of the peak of variation in the refractive index of the sensitive reflecting element 3 related to the desorption of the volatile compound A, allows the amount of volatile compound A adsorbed on the sensitive layer 33 during its exposure to the atmosphere to be tested to be determined and thus its concentration in this atmosphere to be deduced. Typically, the area S1 corresponds to the portion containing inclined hatching under this curve 120. Furthermore, the quantification of the desorbed volatile compound B is also obtained via integration of an area S2 placed under the curve 130 and corresponding to the area of the peak of variation in the refractive index of the sensitive reflecting element 3 related to the desorption of the volatile compound B. This integration of the area S2 therefore allows the amount of volatile compound B desorbed to be obtained and therefore its concentration in the atmosphere to be tested to be deduced. This area S2 corresponds to the portion containing vertical hatching under this curve 130. Thus, the quantification of the volatile compound desorbed is obtained via differential mathematical processing of the curves of variation in optical response as a function of the temperature of the electrically conductive layer 35 and therefore as a function of the temperature of the sensitive layer 33.

According to one alternative (not shown here) the amount of volatile compounds desorbed is determined by comparing the variation in the refractive index before and after heating the sensitive layer 33. According to this alternative, the amount of volatile compounds desorbed is proportional to the equation:

N2−N1
with:
   N1: the value of the refractive index of the sensitive reflecting element 3 with the volatile compound adsorbed, and
   N2: the value of the refractive index of the sensitive reflecting element without the volatile compound.

Desorption temperatures of certain volatile compounds for a given sensitive layer:

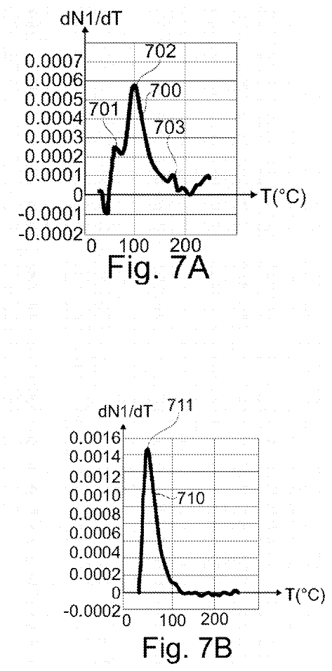

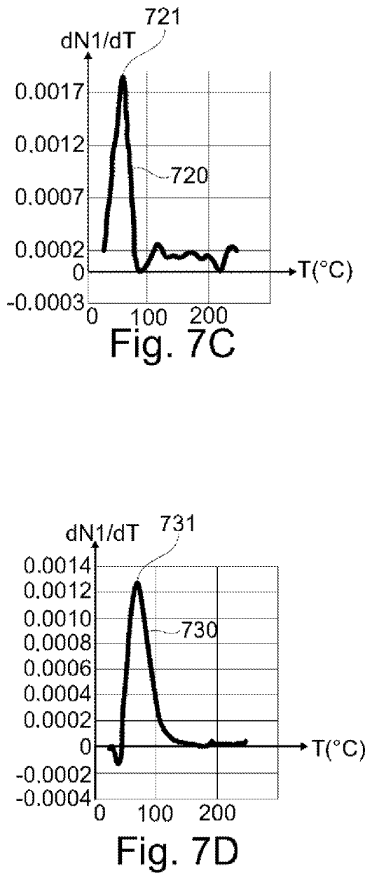

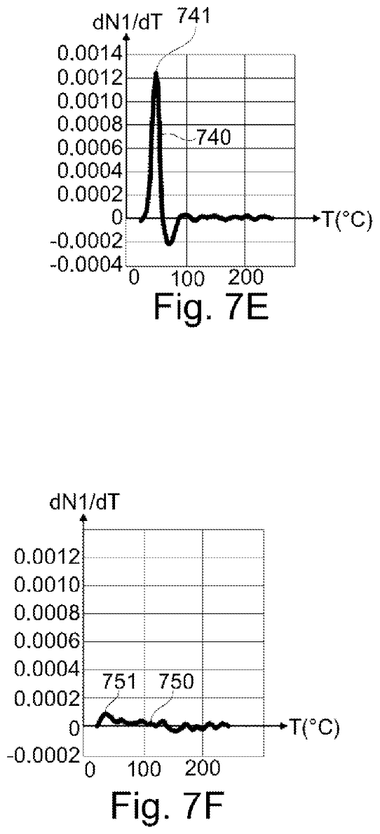

In FIGS. 7A to 7F, various curves illustrating the variation in refractive index of the sensitive reflecting element 3 as a function of the temperature of this sensitive layer 33 have been shown. In these various examples, the sensitive layer 33 has the same chemical composition in all the various experiments and the volatile compound to which this sensitive layer 33 is exposed is known.

The sensitive layer 33 used to demonstrate the difference in desorption temperature as a function of the adsorbed volatile compound A, B, C has a composition S3M3P3.

For this composition, the sensitive layer is produced using a sol-gel formulation composed of 0.33 TEOS (tetraethyl orthosilicate); 0.66 Ph-TEOS (phenyl-triethoxysilane); 7.8 H₂O; 0.07 HCl; and 6.2 PrOH (isopropanol), the various indicated values corresponding to molar proportions.

More particularly, the curves 700, 710, 720, 730, 740 and 750, shown in FIGS. 7A to 7F, respectively, plot the variation in the refractive index of the sensitive reflecting element 3 of composition S3M3P3 as a function of the temperature of this sensitive layer 33, when it is been exposed individually and respectively to the following volatile compounds:
hexane (FIG. 7A),
toluene (FIG. 7B),
acetylacetone (FIG. 7C),
chloroform (FIG. 7D),
ethylene glycol (FIG. 7E), and
water (FIG. 7F).

Each of these curves 700 to 750 comprises one (or more than one) more or less marked peak of variation in the refractive index of the sensitive reflecting element 3. In particular, for water, the peak of variation 751 is very small. This peak of variation 751 is detected at a temperature of the sensitive layer 33 of about 40° C. This spectral analysis allows it to be deduced that the sensitive layer 33 of composition S3M3P3 has a hydrophobic character because it collects very little water and the water desorbs very easily (desorption temperature of 40° C.).

For the other volatile compounds A, B, C, which for their part are of organic nature, the variations in refractive index of the sensitive reflecting element 3 are more marked.

In the curve 710 of FIG. 7B, a marked peak of variation 711 occurs at a temperature of the sensitive layer 33 of about 53° C. corresponding to desorption of the toluene from the sensitive layer 33.

In the curve 720 of FIG. 7C, a marked peak of variation 721 occurs at a temperature of the sensitive layer 33 of about 59° C., corresponding to desorption of the acetylacetone from the sensitive layer 33.

In the curve 730 of FIG. 7D, a marked peak of variation 731 occurs at a temperature of the sensitive layer 33 of about 75° C., corresponding to desorption of the chloroform from the sensitive layer 33.

In the curve 740 of FIG. 7E, a marked peak of variation 741 occurs at a temperature of the sensitive layer 33 of about 53° C., corresponding to desorption of the ethylene glycol from the sensitive layer 33.

The temperatures corresponding to these various peaks correspond to the temperatures at which this curve has a derivative of zero. It is thus possible to easily determine the nature of the desorbed volatile compound depending on its temperature of desorption from the sensitive layer 33. These values may, for example, allow a table to be generated for various volatile compounds A, B, C in order to know their temperature of desorption from the sensitive layer 33 and to facilitate their identification for a sensitive layer 33 of given chemical composition.

The curve 700 of FIG. 7A comprises a first peak 701, a second peak 702 and a third peak 703 of variation in the refractive index of the sensitive layer 33. These first, second and third peaks of variation 701, 702 and 703 correspond to the differential desorption of various isomers of hexane and occur at different temperatures, namely 69° C., 100° C. and 179° C., respectively. These various isomers of hexane may correspond to n-hexane, to 2-methylpentane and to 3-methylpentane, for example. Thus, it is possible to differentiate between various isomers of a given chemical compound depending on their temperatures of desorption from the sensitive layer 33.

Returning to the curves 710 and 740 shown in FIG. 7B and 7E, respectively, it may be seen that toluene and ethylene glycol have the same desorption temperature for the sensitive layer 33 of composition S3M3P3. This sensitive layer 33 of composition S3M3P3 is therefore not of a nature to allow these volatile compounds to be detected if they are in mixture in the atmosphere to be tested, because their peaks of variation 711 and 741 overlap. In such a situation, it may be desirable to use a sensitive reflecting element 3 comprising a plurality of sensitive layers 33a, 33b, 33c (as shown in FIG. 3B) in order notably to allow these two volatile compounds to be discriminated between. This discrimination may be achieved either by using sensitive layers 33a, 33b, 33c having chemical affinities that are perpendicular for these two volatile compounds, or even sensitive layers 33a, 33b, 33c for which these two volatile compounds have different desorption temperatures, as is described below.

### Effect of the chemical composition of the sensitive layer:

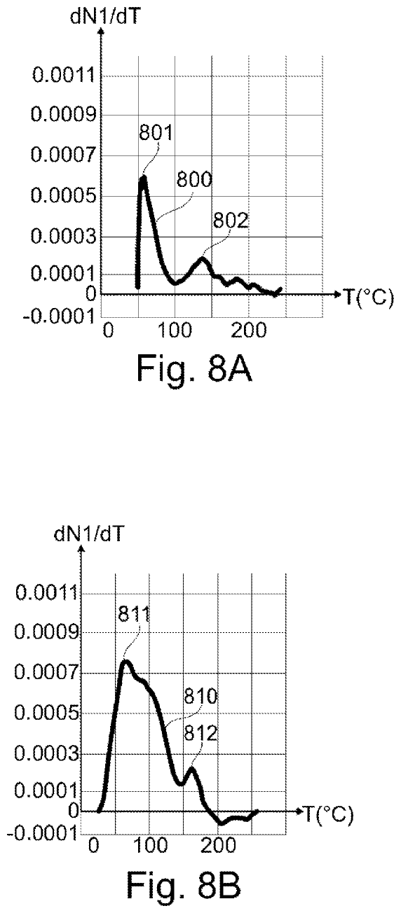

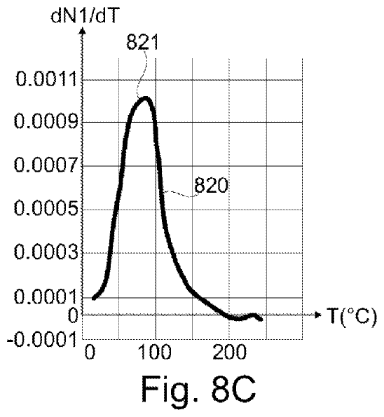

In order to prove the difference in the desorption temperatures of the volatile compounds A, B, C as a function of the chemical composition of the sensitive layer 33 and to make allowance for a situation in which various volatile compounds desorb from the sensitive layer 33 at the same temperature, as was the case for toluene and ethylene glycol in the preceding example, the inventors have exposed sensitive layers 33 of various chemical compositions to the same atmosphere comprising volatile compounds A, B, C. The results of analyses of thermal desorption are shown in FIGS. 8A to 8C. More particularly, the inventors have prepared three sensitive layers of various chemical compositions, named: REF, S3P7 and S3M3P3. These various sensitive layers were produced from sol-gel formulations of the following chemical compositions: the numerical values correspond to molar proportions,

Composition REF: 1 TEOS (tetraethyl orthosilicate); 4 H₂O; 0.17 HCl; 5.8 PrOH (isopropanol);

Composition S3P7: 0.33 TEOS (tetraethyl orthosilicate); 0.33 MTEOS (triethoxymethylsilane); 0.33 Ph-TEOS (phenyl-triethoxysilane); 7.8 H₂O; 0.07 HCl; 6.2 PrOH (isopropanol);

Composition S3M3P3: 0.33 TEOS (tetraethyl orthosilicate); 0.66 Ph-TEOS (phenyl-triethoxysilane); 7.8 H₂O; 0.07 HCl; 6.2 PrOH (isopropanol).

Furthermore, the atmosphere to which these various sensitive layers 33 were simultaneously exposed, for a time of 5 days, corresponded, in this particular example, to the passenger compartment of a motor vehicle.

More precisely, FIG. 8A shows the curve 800 of variation in the refractive index of the sensitive reflecting element 3 having the sensitive layer 33 of composition REF, FIG. 8B shows the curve 810 of variation in the refractive index of the sensitive reflecting element 3 having the sensitive layer 33 of composition S3P7, and FIG. 8C shows the curve 820 of variation in the refractive index of the sensitive reflecting element 3 having the sensitive layer 33 of composition S3M3P3. More particularly, these curves 800, 810 and 820 correspond to the variations in the refractive index of the sensitive reflecting element 3 as a function of the temperature of the sensitive layer 33.

In these various figures, FIGS. 8A to 8C, it may be seen that the curve 820 of FIG. 8C comprises a single highly marked peak of variation 821 in the refractive index of the sensitive reflecting element 3 at a temperature of the sensitive layer 33 of about 86° C. whereas the other curves comprise two peaks of variation 801, 802, 811, 812 in the refractive index of the sensitive reflecting element 3 as a function of temperature. The sensitive layer 33 of composition S3M3P3 may thus be considered to adsorb a single volatile compound, whereas the sensitive layers 33 of compositions REF and S3P7 adsorb two volatile compounds present in the atmosphere of this motor-vehicle passenger compartment. This demonstrates a selectivity of the sensitive layer 33, depending on its chemical composition, to certain volatile compounds A, B, C.

Moreover, the curve 800 of FIG. 8A comprises two peaks of variation 801, 802 in the refractive index of the sensitive reflecting element 3 at temperatures of the sensitive layer 33 of about 55° C. and of about 132° C., respectively, corresponding to the desorption of two volatile compounds from the sensitive layer 33 of chemical composition REF, respectively. Furthermore, the curve 810 of FIG. 8B comprises two peaks of variation 811, 812 in the refractive index of the sensitive reflecting element 3 at temperatures of the sensitive layer 33 of about 70° C. and about 160° C., respectively, corresponding to the desorption of two volatile compounds from the sensitive layer of chemical composition S3P7, respectively. It may be assumed that these two peaks of variation 801, 802, 811, 812 in the refractive index of the sensitive layers 33 shown in FIGS. 8A and 8B correspond to the desorption of two identical volatile compounds A, B. The variation in the desorption temperature of these volatile compounds as a function of the chemical composition of the sensitive layer 33 allows an additional dimension to be added to the selectivity of the optical detecting device 1 combining a plurality of sensitive layers 33a, 33b, 33c as described with reference to FIG. 3B. It is thus possible to carry out more sensitive measurements and to allow these volatile compounds A, B, C to be discriminated between if they have the same temperature of desorption from a sensitive layer 33 of unique composition, as is the case for toluene and ethylene glycol in the case of the sensitive layer 33 of composition S3M3P3 described above.

The various embodiments described above are nonlimiting examples given by way of illustration. Specifically, it will be entirely within the ability of a person skilled in the art to adapt the heating rate of the sensitive layer 33, the thickness e of the sensitive layer 33, the thickness s of the electrically conductive layer 35, and the composition of the substrate layer 31, of the sensitive layer 33, or of the electrically conductive layer 35 as required without departing from the scope of the present description. Furthermore, a person skilled in the art will be able to use light sources having wavelengths different from those described in the present examples without departing from the scope of the present invention. In addition, the refractive indices of the various layers of the sensitive reflecting element 3 may be modified by a person skilled in the art as required without departing from the scope of the present invention.

Thus, a detector of volatile compounds A, B, C that is simple to use, inexpensive, and effective even at low concentrations of volatile compounds A, B, C in the atmosphere to be tested and that delivers measurement results in a short time is obtainable by virtue of the optical detecting device 1 described above and more particularly by virtue of the sensitive reflecting element 3 employed in the detecting and quantifying method 100 described above.

1. An optical device for detecting volatile compounds comprising:
a sensitive reflecting element, the reflected light intensity of which varies as a function of the volatile compounds contained in an atmosphere to be tested and on their concentration, the sensitive reflecting element comprising:
a substrate,
at least one transparent and porous sensitive layer configured to allow volatile compounds contained in the atmosphere to be tested to adsorb and desorb, and
a non-scattering and transparent electrically conductive layer sandwiched between the substrate layer and the sensitive layer and placed directly in contact with the substrate layer and with the sensitive layer, the electrically conductive layer being configured to allow the sensitive layer to be heated by Joule heating in order to allow the volatile compounds adsorbed on the sensitive layer to desorb,
at least one monochromatic or quasi-monochromatic light source placed to illuminate the sensitive layer at an incident angle,
at least one light detector configured to measure the light intensity reflected by the sensitive reflecting element at a detection angle, and
a computing and processing unit configured to determine the volatile compounds present in the atmosphere to be tested depending on their desorption temperatures and their concentration via variation in the light intensity reflected by the sensitive reflecting element.

2. The optical detecting device of claim 1, wherein the presence of one or more desorbed volatile compounds is detected via comparison of the light intensity reflected by the reflecting element without any volatile compounds adsorbed at a given temperature with the light intensity reflected by the reflecting element just after desorption at the same temperature.

3. The optical detecting device of claim 1, wherein the concentration of the one or more volatile compounds detected in the atmosphere to be tested is determined via integration of an area of peaks in variation of the reflected light intensity corresponding to the desorption of the one or more volatile compounds.

4. The optical detecting device of claim 1, wherein the at least one sensitive layer has a thickness comprised between 50 nm and 2000 nm, notably comprised between 400 nm and 1200 nm, and more particularly between 500 nm and 800 nm.

5. The optical detecting device of claim 1, wherein the at least one sensitive layer has an average pore size smaller than 2 nm.

6. The optical detecting device of claim 1, wherein the at least one sensitive layer has a porosity lower than 25%.

7. The optical detecting device of claim 1, wherein the at least one sensitive layer is made of sol-gel silica or of a xerogel.

8. The optical detecting device of claim 1, wherein the at least one sensitive layer has a refractive index comprised between 1.2 and 1.6, and notably between 1.3 and 1.5, at wavelengths comprised between 400 nm and 1000 nm before its exposure to the atmosphere to be tested.

9. The optical detecting device of claim 1, wherein the at least one sensitive layer has a structure configured to increase the variations in the optical signal.

10. The optical detecting device of claim 1, wherein the sensitive reflecting element comprises one or more sensitive layers.

11. The optical detecting device of claim 1, wherein the incident angle and the detection angle are respectively comprised between 30° and 75° with respect to a normal to the sensitive layer.

12. The optical detecting device of claim 1, wherein the electrically conductive layer has a thickness smaller than or equal to 150 nm, and notably comprised between 70 nm and 90 nm.

13. The optical detecting device of claim 1, wherein the electrically conductive layer has a resistivity comprised between 10⁻⁴ Ω·m and 10⁻⁷ Ω·m so as to allow the sensitive layer to be heated by Joule heating.

14. The optical detecting device of claim 1, wherein the electrically conductive layer is made of a transparent conductive oxide, and notably of indium-doped tin oxide, aluminum-doped zinc oxide, gallium-doped zinc oxide, or fluorine-doped tin oxide.

15. A method for detecting and quantifying volatile compounds in an atmosphere to be tested implementing an optical detecting device for detecting volatile compounds, comprising:
a sensitive reflecting element, the reflected light intensity of which varies as a function of the volatile compounds contained in an atmosphere to be tested and on their concentration, the sensitive reflecting element comprising:
a substrate,
at least one transparent and porous sensitive layer configured to allow volatile compounds contained in the atmosphere to be tested to adsorb and desorb, and
a non-scattering and transparent electrically conductive layer sandwiched between the substrate layer and the sensitive layer and placed directly in contact with the substrate layer and with the sensitive layer, the electrically conductive layer being configured to allow the sensitive layer to be heated by Joule heating in order to allow the volatile compounds adsorbed on the sensitive layer to desorb,
at least one monochromatic or quasi-monochromatic light source placed to illuminate the sensitive layer at an incident angle,
at least one light detector configured to measure the light intensity reflected by the sensitive reflecting element at a detection angle, and
a computing and processing unit configured to determine the volatile compounds present in the atmosphere to be tested depending on their desorption temperatures and their concentration via variation in the light intensity reflected by the sensitive reflecting element,
wherein the method comprises the following steps:
illuminating the sensitive layer at an incident angle with the monochromatic or quasi-monochromatic light source and measuring the light intensity reflected by the sensitive reflecting element,
exposing the sensitive reflecting element to the atmosphere to be tested for a predetermined time so as to allow volatile compounds contained in this atmosphere to be tested to adsorb on the sensitive layer of the sensitive reflecting element,
heating the sensitive layer via the electrically conductive layer in order to allow the volatile compounds to be controllably desorbed from the sensitive layer,
measuring the light intensity reflected by the sensitive reflecting element during the heating step in order to determine a variation in the refractive index of the sensitive reflecting element, and
monitoring the variation in the refractive index of the sensitive reflecting element in order to determine the nature and amount of volatile compound desorbed from the sensitive layer in the heating step, the step of monitoring the variation in the refractive index of the sensitive reflecting element being carried out by the computing and processing unit.

16. The method of claim 15, wherein the step of heating the sensitive layer is carried out via controlled heating at a heating rate comprised between 1° C./s and 20° C./s.

17. The method of claim 15, wherein the nature of the volatile compound desorbed during the heating step is determined via differential mathematical processing of the curves of variation in the optical response as a function of temperature.

18. The method of claim 15, wherein the desorbed volatile compound is quantified via integration of an area of a peak of variation in the light intensity reflected by the sensitive reflecting element corresponding to the desorption of the corresponding volatile compound.

19. The method of claim 15, wherein the volatile compound, in the case where there is a mixture of volatile compounds, is identified via deconvolution of the light intensity reflected by the sensitive reflecting element during the desorption of this gas.
